(19) United States
(12) Patent Application Publication
(10) Pub. No.: US 2019/0380879 A1
(43) Pub. Date: Dec. 19, 2019

(54) MULTI-LAYERED ADHESIVE SUBSTRATE

(71) Applicant: Creative Technology Concepts, LLC, Kansas City, MO (US)

(72) Inventor: Bart Patenaude, Kansas City, MO (US)

(21) Appl. No.: 16/479,104

(22) PCT Filed: Jan. 18, 2018
(86) PCT No.: PCT/US18/14268

§ 371 Date: Jul. 18, 2019

Related U.S. Application Data
(60) Provisional application No. 62/448,064, filed on Jan. 19, 2017.

(51) Int. Cl. A61F 13/02; A61M 25/02; A61M 27/00; A61M 16/04

(52) U.S. Cl. CPC A61F 13/025; A61F 13/0253; A61F 13/0266; A61F 13/0269; A61M 2209/088; A61M 27/00; A61M 16/0488; A61M 2025/0266; A61M 25/02

(57) ABSTRACT

A multi-layered thin film adhesive substrate is disclosed, comprising at least three coextensive layers and an adhesive layer. The thin film substrate is resilient, but remains flexible for accurate and smooth application, in uses such as securement of medical dressings and devices.

### CROSS-REFERENCE TO RELATED APPLICATIONS

The present application claims the priority benefit of U.S. Provisional Patent Application Ser. No. 62/448,064, filed Jan. 19, 2017, entitled MULTI-LAYERED ADHESIVE SUBSTRATE, incorporated by reference in its entirety herein.

### BACKGROUND OF THE INVENTION

### Field of the Invention

The present invention relates to multi-layered thin film adhesive substrates and uses for the same.

### Description of Related Art

Thin films with adhesive such as those used in TEGADERM™ brand products are very useful in medicine. Polyurethane is typically used as the thin film which allows water to pass from the skin through the film to the atmosphere, but blocks out dirt and harmful organisms like bacteria. This breathable nature allows the film to remain adhered to skin, but also remain comfortable. When skin stays moist it becomes macerated. Maceration is uncomfortable and weakens the skins defense against invasive organisms. Because these films are thin they also conform well to the macro and micro contours of the body. This is important for adhesion as well as comfort. This also allows the adhesives to conform to medical devices, such as an IV, that may need to be secured to the body to avoid accidental dislodgement.

However, there is also a down side to these thin films especially if they have adhesive. They lack any rigidity, and thus want to collapse in on themselves (due to gravity and/or static electricity) when removed from the carrier backing, similar to plastic wrap (which in fact relies somewhat on this static cling for its usefulness). However, when the thin film contains adhesive, as in the case of medical tapes and dressings, it then gets stuck to itself and generally cannot be pulled apart without damaging the adhesive layer, or the integrity of the thin film, and must be discarded.

Currently, to address this problem, thin films with adhesive are adhered to a sheet of removable liner that covers all the adhesive. A rigid carrier “frame” is mounted on top of the film's periphery that keeps the film in place and in a somewhat inflexible form for handling so that it won't fold in on itself. In use, the practitioner peels off the liner and places the adhesive sheet down (e.g., on the patient), and then the top frame is peeled off. Notably, because the top frame is intended to maintain the thin film in a more rigid shape to prevent self-sticking, this can also interfere with positioning the thin film over the non-planar contours of the body or other surface. Thus, it is common for air bubbles, ridges, or the like to remain in or on the film after the carrier frame is removed, which interferes with the integrity of the dressing. In addition, if the film has not adequately adhered to the patient or substrate, the process of removing the carrier frame has a tendency to lift up portions of the film, which still remain susceptible to self-sticking. In other words, despite the numerous advantages, existing thin film dressings are difficult to work with and improvements are needed. Further, because the carrier frame is only on the peripheral margins of the thin film, practitioners cannot easily resize, shape, or cut the film to a different size or shape without cutting away the carrier frame, and thereby losing the handling benefits it provides.

### SUMMARY OF THE INVENTION

The present invention is broadly concerned with a multilayered thin film adhesive substrate having opposing front and back major surfaces, and comprising a plurality of laminated or multi-ply layers. The substrate comprises a first thin film layer having an outer edge, an interior major surface, and an exterior major surface presenting the opposing back major surface of the substrate. The substrate further comprises a second thin film layer having an outer edge, an interior major surface, and an exterior major surface presenting the opposing front major surface of the substrate. A resilient, intermediate mesh layer is interposed between the first layer and second layer. The intermediate mesh layer has an outer edge, and is positioned in face-to-face relationship adjacent the respective interior major surfaces of the first and second layers. The substrate further comprises a layer of adhesive having an outer edge and extending across the opposing back major surface in a face-to-face relationship. The layers are coextensive with each other, such that the outer edges of each of the layers is in alignment around the perimeter of the substrate.

Methods of using the substrate are also described herein, including methods of applying the substrate to a subject's tissue directly or via intermediate underlayments. The substrate can also be applied to a dressing or medical device to secure it to the subject. The substrate may also be used for bandaging, antimicrobial therapy, or topical chemo therapy, and may be combined with additional active agents for delivery to skin tissue, such as pain relief medications, analgesics, anti-inflammatory medications, steroidal medications, or even being impregnated with bismuth, lead, or the like to shield the applied area against radiation, for example when using targeted X-Ray therapy.

### BRIEF DESCRIPTION OF THE DRAWINGS

FIG. 1 is a perspective view of the multi-layered substrate in accordance with embodiments of the invention;

FIG. 2 is an exploded view of the multi-layered substrate in accordance with embodiments of the invention;

FIG. 3 is a cross-sectional view of the multi-layered substrate in accordance with embodiments of the invention; and

FIG. 4 is an exploded view of the multi-layered substrate in accordance with embodiments of the invention where the second layer comprises openings.

### DETAILED DESCRIPTION

The present invention is concerned with a multi-layered adhesive substrate that comprises a semi-rigid mesh, webbing, or lattice core layer that facilitates easy handling and shaping of the adhesive thin film substrate. The layered construction of the substrate allows much easier handling of these thin films with adhesive. It also allows cutting, resizing, and shaping to any geometry without loss of this ease of handling.

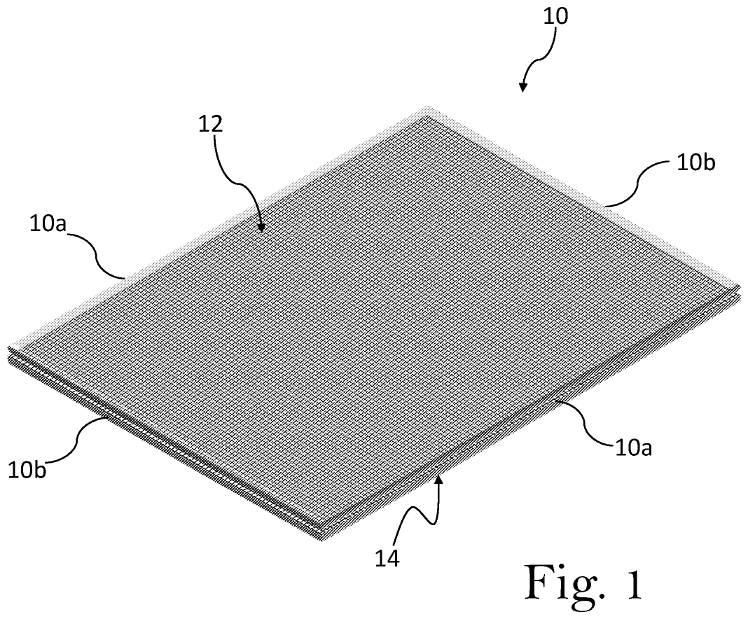

With reference to FIG. 1, the substrate 10 comprises first 12 and second 14 opposing major surfaces, referred to herein as a front surface 12 and a back surface 14, respectively, and outer edges 10a, 10b (which define the perimeter of the substrate). The average total thickness of the substrate 10 will generally range from about 1 mil (0.001 inch) to about 10 mil (0.010 inch), preferably from about 1 mil to about 5 mil, and more preferably from about 2 mil to about 5 mil. The thickness of the substrate 10 preferably has little variation across the expanse of the substrate 10. In other words, the thickness of the substrate 10 is preferably substantially uniform as measured from the front surface 12 to the back surface 14 edge-to-edge.

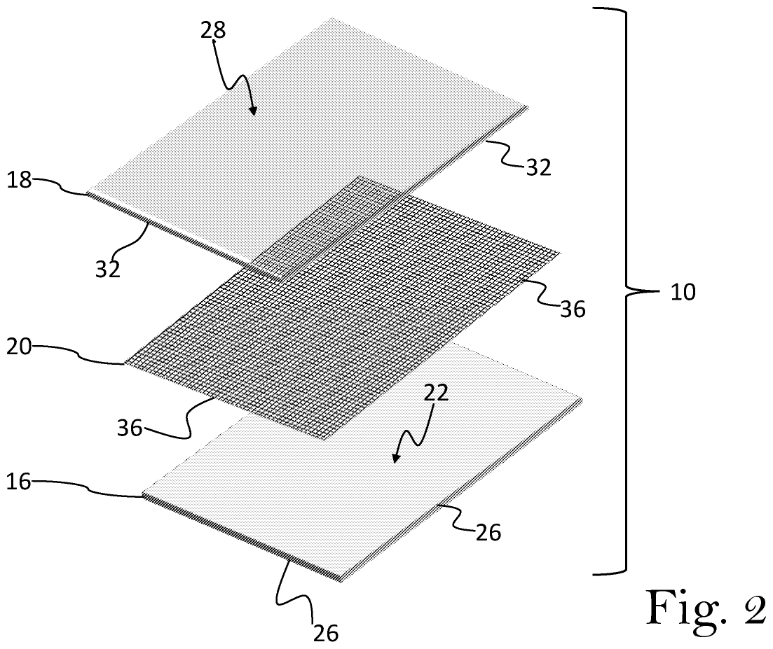

The front surface 12 of the substrate 10 generally remains exposed to the environment, and is preferably free of any adhesives. The back surface 14 of the substrate 10 is configured to engage or contact an underlying surface, such as tissue. The substrate 10 is attached to the underlying surface via an adhesive (aka glue) interposed between the back surface 14 of the substrate 10 and this underlying surface. In one or more embodiments, the adhesive is provided separately from the substrate 10. The adhesive is dispensed or applied onto the underlying surface, followed by mounting or attaching the substrate 10 to the underlying surface with the back surface 14 of the substrate 10 adjacent (i.e., in face-to-face contact with) the adhesive. In one or more embodiments, the back surface 14 of the substrate 10 comprises an adhesive pre-mounted to the back surface 14, which can be in the form of a layer adjacent (i.e., in face-to-face contact with) the back surface 14. In one or more embodiments, the adhesive covers substantially the entire surface area of the back surface 14. In other words, the adhesive extends edge-to-edge over both the central region and the peripheral margins of the back surface 14, such that the adhesive layer 15 (see FIG. 2) outer edges are coextensive (in alignment) with the outer edges of the back surface 14.

Suitable adhesives include pressure sensitive adhesives, as well as heat- or radiation-activated adhesives. It will be appreciated that the appropriate adhesive will depend upon the ultimate intended use of the substrate 10. For example, if the substrate 10 will be topically applied to skin, the adhesive should have good initial tack and long-term adhesion, and be able to tolerate the presence of moisture without releasing. Pressure-sensitive adhesives generally comprise elastomers that are inherently tacky or include tackifying resins. Exemplary adhesives for use in the invention include rubber elastomers, acrylics, acrylates (e.g., cyanoacrylates), epoxies, silicones, zinc oxides (or other hot melt adhesives), solvent-based adhesives, solvent-free adhesives, photosensitive adhesives, hydrocolloids, hydrogels, polyurethanes, styrene block co-polymers, and the like. In some embodiments, the adhesive is biocompatible. Numerous types of suitable adhesives are known in the art, and are commercially-available from suppliers such as 3M, Loctite (e.g., Duro-Tak), Adhesives Research, Ashland Chemical (e.g., Aroset), Dow (e.g., Robond), Polymer Science (e.g., Sofsil, Neosil), DuPont, and ITW Formex. Particularly preferred adhesives will have strong tissue adhesion properties, and adhesives suitable for use in medical tapes and dressings are preferred in some embodiments of the invention. Low trauma adhesives can also be used.

The substrate 10 is generally a continuous flat/planar and flexible/bendable, but non-elastic (aka inelastic) body. In other words, the substrate 10 can bend and conform in all directions to underlying surfaces to which the substrate 10 may be applied, such as for example, conformable topical application to human or animal skin. However, the substrate 10 is preferably not elastic and does not stretch or extend from a relaxed/resting position to an extended position any appreciable degree. In one or more embodiments, materials used in the substrate 10 are preferably non-metallic. In other words, preferred embodiments of the substrate 10 are free of any metals. The substrate 10 can be provided in various shapes and sizes, including an elongated sheet, rectangular, circular, triangular, or other shape. For example, the substrate 10 can have a pair of laterally-spaced longitudinal side edges 10a and a pair of laterally spaced transverse ends 10b, defining a substantially rectangular or square shape, as depicted in FIG. 1. In other embodiments, the substrate 10 can be provided with any other geometric shape, such as having an annular lateral edge defining a substantially circular shape or three lateral edges forming a triangular shape (not shown).

The substrate 10 can be provided in its useable configuration as one or more sheets or strips, each with a releasable liner (not shown) covering the adhesive (when present) to prevent indiscriminate adherence or sticking of the substrate 10 to an undesired surface and/or preserve the adhesive properties of the adhesive layer 15 and/or protect the adhesive layer 15 from attracting dirt, debris, and the like until use. In some embodiments, the substrate 10 can be an elongated strip wound about a spool or dispensing apparatus. In one or more embodiments, the substrate 10 is provided as a large sheet that can be cut down to the necessary size. For example, dimensions such as 30 cm×30 cm are envisioned.

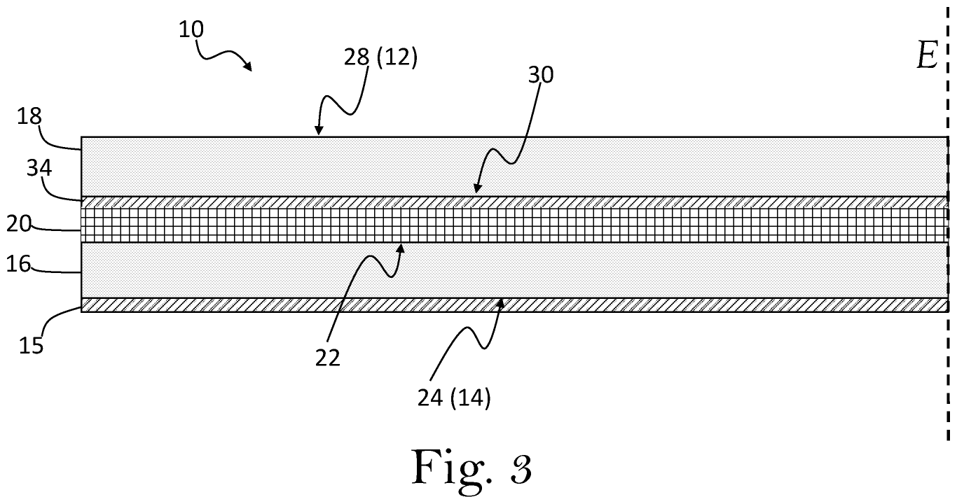

As noted above, and illustrated in the exploded view in FIG. 2, the substrate 10 thin film body is multi-layered (aka multi-ply or laminated) and comprises at least three layers (preferably not including adhesive layers), comprising a first layer 16 (aka bottom layer), a second layer 18 (aka top layer), and an intermediate mesh or webbing layer 20 embedded between the first layer 16 and second layer 18. The layers may be annealed (i.e., subjected to elevated bonding temperature), or not annealed (e.g., bonded through room temperature adhesive). A cross-sectional image of the layers is depicted in FIG. 3. It should be noted that the cross-section layers in FIG. 3 have been enlarged for illustration and clarity, and the respective thicknesses should not be taken as limiting on the invention.

The first layer 16 comprises a first major surface 22 (aka interior major surface 22) and a second major surface 24 (aka exterior major surface 24) and an outer edge(s) 26 defining the perimeter of the layer 16. It will be appreciated that the exterior (second) major surface 24 of the first layer 16 corresponds to the back surface 14 of the substrate 10. The second layer 18 likewise comprises a first major surface 28 (aka exterior major surface 28) and a second major surface 30 (aka interior major surface 30) and an outer edge(s) 32 defining the perimeter of the layer 18. It will be appreciated that the exterior (first) major surface 28 of the second layer 18 corresponds to the top surface 12 of the substrate 10. In general, the mesh layer 20 is embedded in an intermediate layer of adhesive 34 between the first 16 and second 18 layers.

The mesh layer 20 is likewise in a planar configuration with the mesh or webbing extending between outer edges 36 defining the perimeter of the layer 20. Importantly, the outer edges 26, 32, and 36, of each of the first layer 16, second layer 18, and intermediate mesh layer 20 are coextensive (in alignment). This means that the outer edges of the layers each terminate in alignment with one another, such as along a shared (vertical) plane E that runs perpendicular to the plane lateral of the substrate 10 as illustrated in FIG. 3. In other words, the first 16 and second 18 layers are laminated with respective interior major surfaces 22, 30 in a face-to-face relationship with the intermediate mesh layer 20, and the intermediate mesh layer 20 extending along the entire (i.e., coextensive with) surface area of each of the interior major surfaces 22, 30. It will be appreciated that the mesh or webbing layer 20 will thus be “embedded” between the first layer 16 and second layer 18, preferably in an intermediate adhesive layer 34, as depicted in FIG. 3. The intermediate adhesive layer 34 can be provided as part of the first layer 16, and applied or pre-formed on the interior major surface 22 of the first layer 16. Alternatively, the intermediate adhesive layer 34 can be provided as part of the second layer 18, and applied or pre-formed primarily on the interior major surface 30 of the second layer 18, as depicted in FIG. 3. Any suitable adhesive material can be used for intermediate adhesive layer 34, including materials described herein. It will be appreciated that since the intermediate adhesive layer 34 remains interior to the film 10, it does not necessarily have to be compatible for skin contact, and a wider variety of suitable (and preferably permanent) adhesives may be used.

Exemplary materials for the first 16 and/or second 18 layer include suitable thin film materials, such as polyurethane, polyethylene, polypropylene, polyamide, polytetrafluoroethylene, polystyrene, silicones, rubbers, latex, rayon, composites thereof, copolymers thereof, combinations thereof, and the like. The first 16 and/or second 18 layer may itself be a laminated or extruded sheet, or may comprise woven or nonwovent fibers. In some embodiments, the first 16 and/or second 18 layer materials are preferably biocompatible. Preferably, the materials are water resistant, but “breathable” in that they allow moisture to pass through the layer, while excluding dirt, bacteria, viral particles, and the like. Preferably, the materials selected for the first 16 and/or second 18 layers are translucent, transparent, or substantially visually transparent, with a percent visible light transmittance of at least 50%.

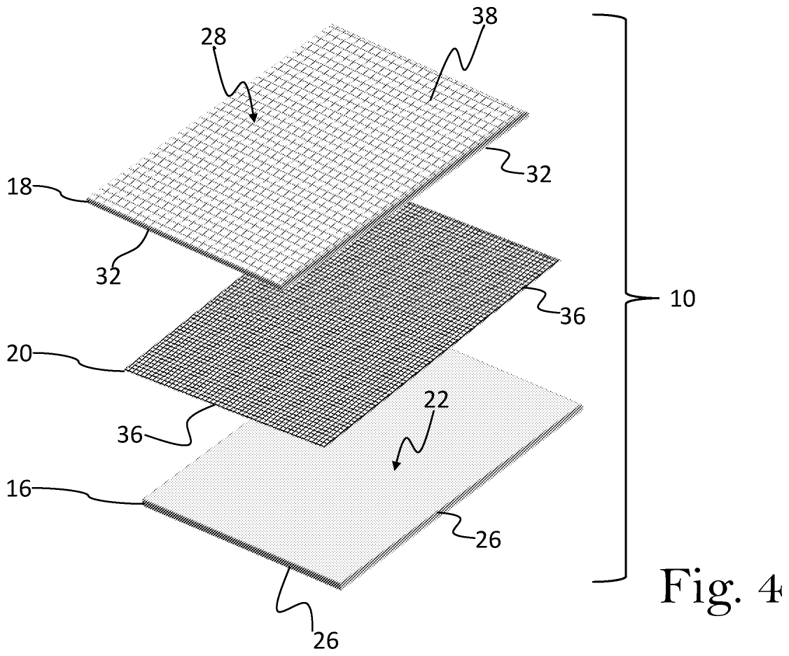

Notably, each of the first 16 and/or second 18 layers can be made of the same material, or a different material may be selected for each of the first layer 16 and second layer 18, without departing from the scope of this invention. In some embodiments, the physical configuration of the first layer 16 can be different from the second layer 18. For example, in some embodiments, the first layer 16 is substantially solid, whereas the second layer 18 comprises a plurality (e.g., pattern) of holes, voids, or openings 38 formed through the thickness of the layer 18 across the first 28 and second 30 major surfaces, as depicted in FIG. 4. These apertures 38 in the second layer 18 permit egress of moisture and contribute to the “breathability” of the multi-layered substrate 10. The pattern and/or density of the openings 38 in the second layer 18 can be adjusted to allow more or less fluid to pass and to make the substrate 10 more flexible and have more of the properties of a single layer film with adhesive. In general, the size of the openings 38 will range from about 1 micron to about 10 mm, or from about 100 microns to about 5 mm, or from about 500 microns to about 5 mm, or from about 1 mm to about 5 mm, depending upon the embodiments.

The material thickness of each of the first 16 and/or second 18 layers is preferably from about 0.5 mil to about 5 mil, preferably from about 0.5 mil to about 2.5 mil, and more preferably from about 0.5 mil to about 2 mil The thickness of each of the first 16 and/or second 18 layers preferably has little variation across the expanse of the substrate 10. In other words, the thickness of the substrate 10 is preferably substantially uniform as measured between the first and second major surfaces edge-to-edge (it being appreciated that this thickness will vary in portions of the second layer 18 containing openings, as discussed above).

As noted above, in certain embodiments, at least one (and preferably only one) major surface of the first 16 and/or second 18 layers each comprises an adhesive (or layer of adhesive) pre-mounted to the surface, which can be in the form of a layer adjacent 34 (i.e., in face-to-face contact with) the one major surface. In one or more embodiments, the adhesive covers substantially the entire surface area of the major surface (e.g., from edge to edge). In some embodiments, both major surfaces 22, 24 of the first layer 16 may be covered with an adhesive layer (such that an additional layer of adhesive is adjacent the mesh or webbing layer 20, not depicted). Alternatively, the thickness and material of the intermediate adhesive layer 34 is such that the mesh layer 20 is embedded in the adhesive layer (not shown) between the first 16 and second 18 layers of the substrate 10.

In one or more embodiments, the intermediate mesh layer 10 comprises polypropylene or other synthetic (e.g., plastic) netting or mesh, such as nylon, polyethylene, polyester, and the like. In general, synthetic mesh or netting materials with approximately 4-mm square openings are preferred, although other configurations are suitable. Monofilament synthetic woven or nonwoven fibers can be used with varying fiber size (diameter), thread count, and hole size. The mesh, webbing, or lattice configuration is preferably uniform across the expanse of the layer 20 for example as in a grid or matrix pattern. In other words, the density (number) of openings in a first portion of the layer 20 will be substantially equivalent to the density of openings in a second, different portion of the layer 20 having the same surface area. The mesh or webbing may also be knitted mesh or non-knitted sheet forms, woven or nonwoven fibers, and the like. The material selected may be absorbent, non-absorbent, absorbable, non-absorbable or a combination of absorbable and non-absorbable materials. In some cases, the mesh layer 20 can even be a porous mat or web spun from fibers in an irregular pattern. Regardless of the embodiment, the mesh or webbing material should be selected to provide a flexible but resilient skeleton, scaffolding, or core that maintains an amount of rigidity to the thin film layers (first 16 and second 18 layers) and facilitates easy handling and shaping of the substrate 10, while allowing the substrate 10 to still conform to the skin. As such, the substrate 10 does not fold in on itself under gravity with the liner is removed, and is not susceptible to static cling.

A significant advantage of the inventive substrate 10 is that it is severable. As used herein, the term “severable” means that the substrate 10 can be cross-sectionally cut into one or more pieces that conform to the desired shape without losing functionality. In other words, the term is directed towards cutting through the substrate 10 along a plane that cuts across (and is preferably generally perpendicular to) the multi-layers, and does not encompass separating the substrate 10 along the plane defined by the front and back surfaces (for example, pulling apart layers in the multi-layered substrate 10). The substrate 10 can be cut or severed using conventional scissors, medical shears, utility knives, scalpels, and the like. In some embodiments, the substrate 10 may be tearable. The substrate 10 could also be provided with perforations that can be torn.

In one or more embodiments, medications, antimicrobials (chlorhexidine), anti-fungals, and other active agents for delivery to the patient (e.g., topical delivery to skin tissue) and preventing infection on the skin can be included in the substrate 10 as part of the adhesive layer(s), the film itself, or as pockets of medication (e.g., imbedded time release capsule or the like). See US20110290259, filed Jan. 7, 2010, for an example of chlorhexidine-containing antimicrobial laminates, incorporated by reference herein to the extent it does not contradict the present disclosure. Medications include pain relief medications, analgesics, anti-inflammatory medications, steroidal medications, and the like.

The multi-layered adhesive substrate 10 is particularly suited for use in medical (or veterinary) applications, such as a medical/veterinary tape, for fixation of medical/veterinary devices, and/or fixation of primary dressings. However, non-medical uses of the improved thin film substrate 10 are also envisioned, such as an improved utility or craft tape. In medical or veterinary uses, the substrate 10 is non-implantable, and suited primarily for topical (external) applications. Thus, the materials used in the substrate 10, and particularly the first 16 and second 18 layers (i.e., the external layers) are preferably non-absorbable, non-resorbable, non-degradable, water-proof or at least moisture resistant, and are not suitable or compatible for implantation into a subject's body.

In use, the substrate 10 can be provided as one or more sheets or strips, each with a releasable liner covering and removably attached to the adhesive layer to prevent indiscriminate adherence or sticking of the substrate 10 to an undesired surface and/or preserve the adhesive properties of the adhesive layer and/or protect the adhesive layer from attracting dirt, debris, and the like until use. In some embodiments, the substrate 10 can be an elongated strip wound about a spool or dispensing apparatus. In one or more embodiments, the substrate 10 is provided as a large sheet that can be cut down to the necessary size.

As noted above, prior to application, the substrate 10 may first need to be released from the releasable liner to expose the adhesive layer 15, when pre-mounted onto the substrate 10. In some embodiments, to enhance adhesion, the tissue of the subject (e.g., human, non-human animal, etc.) can be prepared before applying the substrate 10 or adhesive. Suitable skin preparation techniques include hair removal, and/or wiping the skin around and near a wound area, or other area where the substrate 10 will be adhered, with isopropyl alcohol (e.g., Preppies Skin Barrier Wipes; Kendall), betadine, chlorhexidine, and the like. The substrate 10 is then applied (and adhered) to the subject's tissue with the adhesive layer adjacent the tissue. Even when it is removed from the liner, the substrate 10 resists gravity and static cling and does not fold in on itself. The ease of handling of the substrate 10 facilitates accurate and smooth application of the substrate 10 so that no ridges, air bubbles, or creases are present in the applied substrate 10. The lack of a separate rigid frame also permits the flexible substrate 10 to be applied smoothly over contours of the underlying body structures without gapping or creasing. The substrate 10 is useful for human therapeutic applications, as well as in veterinary applications for animals, such as dogs, cats, horses, rodents, primates, avian species, as well as for basic research.

The substrate 10 can be used for fixation and/or securement of primary dressings (e.g., cotton, gauze, etc.), as well as any number of medical devices, such as IV lines, tracheal tubes, nasogastric tubes, ventilators, drains, catheters, central lines, arterial lines, and the like (not shown). In such use, a first portion of a piece (or length) of substrate is adhered to the dressing or device, and a second portion of the substrate is adhered to the subject to thereby removably affix the dressing or device to the patient in a relatively secured position that resists dislodgement. It will be appreciated that the substrate may also be first adhered to the subject before being affixed to the device or dressing, or may be simultaneously applied to both. The substrate can be removed by peeling a portion of an edge of the substrate back from the dressing/device and/or the subject's tissue, followed by careful peeling/removal of the entire substrate to avoid tissue damage. Depending upon the type of adhesive used, solvents can be used to facilitate removal, such as acetone, oils (e.g., paraffin oil), and/or saline.

In some embodiments, the methods of use further include an underlayment or intermediate layer (not shown) to which the substrate is applied, instead of being applied directly to the subject's tissue. In such an approach, the underlayment is first secured to the subject's skin, and the substrate is then applied to the underlayment and adheres the underlayment, rather than directly to the tissue of the subject. An example of this type of system is Rigid™ strapping tape, which is typically used for athletic immobilization.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

While the drawings illustrate, and the specification describes, certain preferred embodiments of the invention, it is to be understood that such disclosure is by way of example only. Embodiments of the present invention are described herein with reference to cross-section illustrations that are schematic illustrations of idealized embodiments of the present invention. As such, variations from the shapes of the illustrations as a result, for example, of manufacturing techniques and/or tolerances, are to be expected. Unless noted, there is no intent to limit the principles of the present invention to the particular disclosed embodiments. For example, in the drawings, the size and relative sizes of layers and regions may be exaggerated for clarity. In addition, embodiments of the present invention should not be construed as limited to the particular shapes of regions illustrated herein but are to include deviations in shapes that result, for example, from manufacturing. For example, the device illustrated as a rectangle may have rounded or curved features. Furthermore, directional references (e.g., top, bottom, front, back, up, down, etc.) are used herein solely for the sake of convenience with reference to the figures, and should be understood only in relation to each other. For instance, a component might in practice be oriented such that surfaces referred to as “top” and “bottom” are sideways, angled, inverted, etc. relative to the chosen frame of reference.

1. A multilayered thin film adhesive substrate having opposing front and back major surfaces, and comprising:
a first layer having an outer edge, an interior major surface, and an exterior major surface presenting said opposing back major surface of said substrate;
a second layer having an outer edge, an interior major surface, and an exterior major surface presenting said opposing front major surface;
a resilient, intermediate mesh layer between the first layer and second layer, said intermediate mesh layer having an outer edge, and being in face-to-face relationship adjacent said respective interior major surfaces; and
a layer of adhesive having an outer edge, and being in face-to-face relationship adjacent said opposing back major surface,
wherein the outer edge of each of said layers is in alignment.

2. The substrate of claim 1, further comprising an intermediate adhesive layer between said first layer and said second layer adjacent said intermediate mesh layer.

3. The substrate of claim 1, wherein said substrate is flexible and inelastic.

4. The substrate of claim 1, wherein said substrate is non-implantable.

5. The substrate of claim 1, wherein said substrate has a thickness of from about 1 mil to about 10 mil.

6. The substrate of claim 1, wherein said substrate is severable.

7. The substrate of claim 1, further comprising a releasable liner in face-to-face relationship adjacent said adhesive layer.

8. The substrate of claim 1, wherein said first layer or second layer comprises a material selected from the group consisting of polyurethane, polyethylene, polypropylene, polyamide, polytetrafluoroethylene, polystyrene, silicone, rubber, latex, rayon, composites thereof, copolymers thereof, and combinations thereof.

9. The substrate of claim 1, wherein the adhesive layer comprises a material selected from the group consisting of rubber elastomers, acrylics, acrylates, epoxies, silicones, hot melt adhesives, solvent-based adhesives, solvent-free adhesives, photosensitive adhesives, hydrocolloids, hydrogels, polyurethanes, styrene block co-polymers, and combinations thereof.

10. The substrate of claim 1, wherein said intermediate mesh layer comprises polypropylene, nylon, polyethylene, or polyester netting.

11. The substrate of claim 1, wherein said intermediate mesh layer comprises woven or nonwoven fibers.

12. The substrate of claim 1, wherein said second layer comprises a plurality of openings formed therein, said openings extending through a thickness of said second layer.

13. A method of securing a dressing or device to a subject, said method comprising, providing a multilayered thin film adhesive substrate according to claim 1, applying said substrate to said subject wherein a portion of said adhesive layer is adjacent a tissue of said subject, and applying said substrate to said dressing or device wherein a portion of said adhesive layer is adjacent said dressing or device.

14. The method of claim 13, wherein said device is selected from the group consisting of IV lines, tracheal tubes, nasogastric tubes, ventilators, drains, catheters, central lines, and/or arterial lines.

15. The method of claim 13, wherein said substrate comprises a releasable liner in face-to-face relationship adjacent said adhesive layer, said method further comprising removing said releasable liner prior to applying said substrate.

16. The method of claim 15, wherein said substrate does not fold in on itself after removing said liner.

17. The method of claim 13, wherein said substrate is smooth and non-creased after said applying.

18. The method of claim 13, wherein said substrate is topically applied directly to said subject's tissue or to an intermediate underlayment applied to said tissue.

19. The method of claim 13, wherein said substrate is not implantable in said subject.

20. The method of claim 13, further comprising removing said substrate from said subject, said method comprising peeling an edge of said substrate to release said adhesive layer.
